SEQUENCE LISTING (6 sequences)

SEQ ID NO: 1 - protein, 269 aa, Thermomyces lanuginosus [sequence omitted]

SEQ ID NO: 2 - protein, 269 aa, synthetic construct
  REGION 1..269: note = Variant
EVSQDLFNQFNLFAQYSAAAYCGKNNDAPAGTNITCTGNACPEVEKADATFLYSFEDSGVGDVTGFLALDNTNKLIVLSFRGSRSIENWIANLNFWLKKINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

SEQ ID NO: 3 - protein, 273 aa, synthetic construct
  REGION 1..273: note = Variant
EVSQDLFNQFNLFAQYSAAAYCGKNNDAPAGTNITCTGNACPEVEKADATFLYSFEDSGVGDVTGFLALDNTNKLIVLSFRGSRSIENWIANLNFWLKKINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFQATDACNAGGF

SEQ ID NO: 4 - protein, 317 aa, synthetic construct
  REGION 1..317: note = Variant
EVSQDLFNQFNLFAQYSAAAYCGKNNDAPAGTNITCTGNACPEVEKADATFLYSFEDSGVGDVTGFLALDNTNKLIVLSFRGSRSIENWIANLNFWLKKINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFQATDACNAGGFSWRRYRSAESVDKRATMTDAELEKKLNSYVQMDKEYVKNNQARS

SEQ ID NO: 5 - protein, 345 aa, Fusarium oxysporum
MLLLPLLSAITLAVASPVALDDYVNSLEERAVGVTTTDFGNFKFYIQHGAAAYCNSEAAAGSKITCSNNGCPTVQGNGATIVTSFGSKTGIGGYVATDSARKEIVVSFRGSINIRNWLTNLDFGQEDCSLVSGCGVHSGFQRAWNEISSQATAAVASARKANPSFKVISTGHSLGGAVAVLAAANLRVGGTPVDIYTYGSPRVGNVQLSAFVSNQAGGEYRVTHADDPVPRLPPLIFGYRHTTPEFWLSGGGGDTVDYTISDVKVCEGAANLGCNGGTLGLDIAAHLHYFQATDACNAGGFSWRRYRSAESVDKRATMTDAELEKKLNSYVQMDKEYVKNNQARS

SEQ ID NO: 6 - DNA, 942 bp, synthetic construct
  misc_feature 1..942: note = Optimized nucleotide sequence
  source 1..942: mol_type = other DNA
cacaagtttgtacaaaaaagcaggctccgcgccaccatgcgcagctcccttgttctgttcttcgtcagcgcgtggacggccttggcctcccctattcgtcgagaggtctcgcaagatctgttcaaccagttcaatctcttcgctcagtattctgcagccgcctactgcggaaagaacaacgacgcccccgctggtaccaacatcacgtgcacgggcaacgcctgccccgaggtcgagaaggcggacgccacgtttctctactcgttcgaggacagcggcgtgggcgatgtcaccggcttcctggctctcgacaacacgaacaagctcatcgtcctctctttccgcggcagccggtccatcgagaactggatcggcaaccttaacttcgacctcaaggagatcaacgacatctgctccggctgccgcggccacgacggcttcacttcgtcctggaggagcgtcgccgacacgctgcgccagaaggtggaggacgctgtgcgcgagcatcccgactaccgcgttgtttttaccggacacagcctcggtggtgcgctcgctactgttgccggagccgacctgcgcggcaatgggtacgacatcgacgtgttcagctatggcgccccccgagtcggaaaccgcgctttcgccgagttcctgaccgtccagaccggcggcactctctaccgcatcacccacaccaacgatattgtccctcgcctccccccgcgcgaattcggttacagccactctagccccgagtactggatcaagtctggcaccctcgtccccgtcacccgaaacgacatcgtgaagatcgagggcatcgatgccaccggcggcaacaaccagcctaacattccggacatccctgcgcacctgtggtacttcggtctgatcggtacctgtctttgagcgcgccgacccagctttcttgtacaaagt

(19) United States
(12) Patent Application Publication
(10) Pub. No.: US 2024/0401011 A1
(43) Pub. Date: Dec. 5, 2024

(54) IMPROVED LIPASE FOR DEFOAMING

(71) Applicant: DANISCO US INC, Palo Alto, CA (US)

(72) Inventors: Sharief BARENDS, OEGSTGEEST (NL); Svetlana Laura IANCU, OEGSTGEEST (NL); Scott D. POWER, PALO ALTO, CA (US); Marco VAN BRUSSEL-ZWIJNEN, OEGSTGEEST (NL)

(21) Appl. No.: 18/808,493

(22) Filed: Aug. 19, 2024

Related U.S. Application Data
(63) Continuation of application No. 17/439,561, filed on Sep. 15, 2021.
(60) Provisional application No. 62/819,029, filed on Mar. 15, 2019.

(51) Int. Cl. C12N 9/20; C12N 1/14

(52) U.S. Cl. CPC C12N 9/20; C12N 1/14; C12Y 301/01003

(57) ABSTRACT

Disclosed are compositions and methods relating to an improved hybrid lipase enzyme for reducing foaming in, for example, a carbohydrate fermentation process.

### FIELD OF THE INVENTION

This application claims priority to U.S. Provisional Patent Application No. 62/819,029 filed Mar. 15, 2019, the disclosure of which is incorporated by reference in its entirety.

Disclosed are compositions and methods relating to an improved hybrid lipase enzyme for reducing foaming in, for example, a carbohydrate fermentation process.

### SEQUENCE LISTING

The sequence listing submitted via Patent Center, in compliance with 37 C.F.R. § 1.52(e), is incorporated herein by reference. The sequence listing XML file submitted via Patent Center contains the file “20240702_NB41556USPCN_SeqLst”, created Jul. 2, 2024, which is 9,885 bytes in size.

### BACKGROUND

Numerous commercial products are produced in fermentation processes utilizing “cell factories,” which are typically microorganisms. In such processes large amounts of foam may be produced, which reduces the effective capacity per unit fermenter volume and may cause fermentation broth to overflow from the fermenter through air vents.

Foaming can be a particular problem in fuel ethanol production using carbohydrate substrates and yeast as a fermenting organism. Foaming appears to be exacerbated when protease is added during or upstream of fermentation.

The use of lipases to reduce foaming in fuel ethanol production has previously been described in, e.g., WO2004029193, WO2008135547 and WO201875430. Nonetheless, the need exists for superior antifoaming enzymes at reduced costs.

### SUMMARY

The present compositions and methods relate to an improved variant lipase polypeptide, and methods of use, thereof. Aspects and embodiments of the present compositions and methods are summarized in the following separately-numbered paragraphs:
1. In a first aspect, a variant Thermomyces lanuginosus lipase having at least 95%, optionally at least 98% and optionally at least 99% amino acid sequence identity to the amino acid sequence of SEQ ID NO: 4 and having improved defoaming activity in a fermentation process compared to a reference lipase having the amino acid sequence of SEQ ID NO: 5 is provided, wherein the variant lipase comprises: substantially the entire contiguous amino acid sequence of T. lanuginosus lipase, including the the N-terminus, having one or more substitutions selected from the group consisting of G91A, D96W and E99K, with reference to SEQ ID NO; 4, the substantially the entire contiguous amino acid sequence of T. lanuginosus lipase existing as a fusion protein with a contiguous amino acid sequence from Fusarium oxysporum lipase having the amino acid sequence of SEQ ID NO: 2, where the variant lipase has, as its C-terminus, at least 12 but fewer than 55 amino acid residues derived from the C-terminus of F. oxysporum lipase, and wherein the variant lipase does not have the amino acid sequence of SEQ ID NO: 3 or SEQ ID NO: 5.
2. In some embodiments, the variant lipase of paragraph 1 has, as its C-terminus, at least 12 but fewer than 15 amino acid residues derived from the C-terminus of F. oxysporum.
3. In some embodiments, the variant lipase of paragraph 1 or 2 has, as its C-terminus, 12 amino acid residues derived from the C-terminus of F. oxysporum.
4. In some embodiments, the variant lipase of any of paragraphs 1-3 has the substitutions G91A, D96W and E99K.
5. In some embodiments, the variant lipase of any of paragraphs 1-4 has a small number of fewer or additional residues at the C-terminus of the contiguous amino acid sequence of T. lanuginosus lipase.
6. In some embodiments, the variant lipase of any of paragraphs 1-4 has a truncation of residues at the C-terminus of the contiguous amino acid sequence of T. lanuginosus lipase.
7. In some embodiments, the variant lipase of any of paragraphs 1-6 has the amino acid sequence of SEQ ID NO: 4.
8. In some embodiments of the variant lipase of any of paragraphs 1-7 the fermentation process in which the variant lipase has improved defoaming activity in simultaneous sachharification and fermentation.
9. In another aspect, an improved method for reducing foaming in an ethanol production process using a carbohydrate substrate as feedstock is provided, comprising adding before or during a fermentation step the variant lipase of any of paragraphs 1-7 having improved defoaming activity in a fermentation process compared to the reference lipase having the amino acid sequence of SEQ ID NO: 5.
10. In some embodiments of the improved method of paragraph 9, the fermentation process is saccharification and/or fermentation.
11. In some embodiments of the improved method of paragraph 9 or 10, the fermentation process is simultaneous sachharification and fermentation.
12. In another aspect, a variant Thermomyces lanuginosus lipase having at least 95%, optionally at least 98% and optionally at least 99% amino acid sequence identity to the amino acid sequence of SEQ ID NO: 4 and having improved expression in a Trichoderma host compared to a reference lipase having the amino acid sequence of SEQ ID NO: 5 is provided, wherein the variant lipase comprises: substantially the entire contiguous amino acid sequence of T. lanuginosus lipase, including the the N-terminus, having one or more substitutions selected from the group consisting of G91A, D96W and E99K, with reference to SEQ ID NO; 4, the substantially the entire contiguous amino acid sequence of T. lanuginosus lipase existing as a fusion protein with a contiguous amino acid sequence from Fusarium oxysporum lipase having the amino acid sequence of SEQ ID NO: 2, where the variant lipase has, as its C-terminus, at least 12 but fewer than 55 amino acid residues derived from the C-terminus of F. oxysporum lipase, and wherein the variant lipase does not have the amino acid sequence of SEQ ID NO: 3 or SEQ ID NO: 5.
13. In some embodiments, the variant lipase of paragraph 12 has, as its C-terminus, at least 12 but fewer than 15 amino acid residues derived from the C-terminus of F. oxysporum.
14. In some embodiments, the variant lipase of paragraph 12 or 13 has, as its C-terminus, 12 amino acid residues derived from the C-terminus of F. oxysporum.
15. In some embodiments, the variant lipase of any of paragraphs 12-14 has the substitutions G91A, D96W and E99K.
16. In some embodiments, the variant lipase of any of paragraphs 12-15 has a small number of fewer or additional residues at the C-terminus of the contiguous amino acid sequence of T. lanuginosus lipase.
17. In some embodiments, the variant lipase of any of paragraphs 12-16 has a truncation of residues at the C-terminus of the contiguous amino acid sequence of T. lanuginosus lipase.
18. In some embodiments, the variant lipase of any of paragraphs 12-17 has the amino acid sequence of SEQ ID NO: 4.
19. In some embodiments or the variant lipase of any of paragraphs 11-18, the fermentation process in which the variant lipase has improved defoaming activity in simultaneous sachharification and fermentation.

These and other aspects and embodiments of the compositions and methods will be apparent from the description and drawings.

### BRIEF DESCRIPTION OF THE DRAWINGS

FIG. 1 is a diagram depicting simplified structures of the lipase molecules described, herein. The dark-colored bars are amino acid sequences derived from Thermomyces lanuginosus lipase (TLL). The grey-colored bars are amino acid sequences derived from of Fusarium oxysporum lipase (FOX).

FIG. 2 is a Coomassie-stained SDS-PAGE gel loaded with samples of the lipase molecules described, herein.

FIG. 3 is a graph showing total protein concentration in the supernatant of culture broth from cells expressing LIP3 (squares), LIP4 (diamonds) and LIP5 (triangles).

FIG. 4 is a graph showing broth lipase activity of LIP3 (squares), LIP4 (diamonds) and LIP5 (triangles).

FIG. 5 is a graph showing the stability of LIP4 (diamonds), LIP5 (triangles) and an unrelated commercially available lipase and truncated variant, thereof (shape 1 and shape 2, respectively). pH is represented by + symbols.

### DETAILED DESCRIPTION

Prior to describing the various aspects and embodiments of the present compositions and methods, the following definitions and abbreviations are described.

### 1. Definitions and Abbreviations

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art. The following terms are provided below.

### 1.1. Abbreviations and Acronyms

The following abbreviations/acronyms have the following meanings unless otherwise specified:
BSA bovine serum albumin
° C. degrees Centigrade
DCW dry cell weight
DNA deoxyribonucleic acid
DS dissolved solids
FFAeq free fatty acid equivalent
g or gm gram
GA glucoamylase
GAU/g ds glucoamylase activity unit/gram dry solids
H₂O water
hr hour
kDa kiloDalton
kg kilogram
M molar
mg milligram
min minute
mL and ml milliliter
mm millimeter
mM millimolar
MW molecular weight
ppm parts per million, e.g., μg protein per gram dry solid
REMI restriction enzyme-mediated integration
SDS-PAGE sodium dodecyl sulfate polyacrylamide gel electrophoresis
sec second
sp. species
SSF simultaneous saccharification and fermentation
TP total protein
Tris-HCl tris(hydroxymethyl)aminomethane hydrochloride
U units
v/v volume/volume
w/v weight/volume
w/w weight/weight
wt % weight percent
μg microgram
μL and μl microliter
μm micrometer
μM micromolar

### 1.2. Definitions

The term “starch” refers to any material comprised of the complex polysaccharide carbohydrates of plants, comprised of amylose and amylopectin with the formula (C₆H₁₀O₅)ₓ, wherein X can be any number. The term includes plant-based materials such as grains, cereal, grasses, tubers and roots, and more specifically materials obtained from wheat, barley, corn, rye, rice, sorghum, brans, cassava, millet, milo, potato, sweet potato, and tapioca. The term “starch” includes granular starch. The term “granular starch” refers to raw, i.e., uncooked starch, e.g., starch that has not been subject to gelatinization.

The terms “lipase” refer to an enzyme that catalyzes the hydrolysis of fats (i.e., lipids). Lipases are a subclass of esterases. As used herein, the term lipase is intended to be interpreted broadly to encompass enzymes classified as EC.3.1.1.X, and especially EC.3.1.1.1 and EC.3.1.1.2.

The term “titratable phospholipase unit (TIPU)” refers to the amount of enzyme that liberates 1 μmol free fatty acid equivalent (FFAeq) per minute at 30° C. and pH 7.0.

The terms “protease” and “proteinase” refer to an enzyme protein that has the ability to perform “proteolysis” or “proteolytic cleavage” which refers to hydrolysis of peptide bonds that link amino acids together in a peptide or polypeptide chain forming the protein. This activity of a protease as a protein-digesting enzyme is referred to as “proteolytic activity.” As used herein, the term lipase is intended to be interpreted broadly to encompass enzymes classified as EC.3.4.X.

The terms “serine protease” refers to enzymes that cleave peptide bonds in proteins, in which enzymes serine serves as the nucleophilic amino acid at the enzyme active site. Serine proteases fall into two broad categories based on their structure: chymotrypsin-like (trypsin-like) or subtilisin-like. These enzymes are classified as EC.3.4.16.

The term “glucoamylase” refers to enzymes claified under EC.3.2.1.3 (glucoamylase, α-1,4-D-glucan glucohydrolase), which remove successive glucose units from the non-reducing ends of starch. These enzymes may also hydrolyze α-1,6 and α-1,3 linkages although at much slower rates than α-1, 4 linkages.

The terms “α-amylase” refer to enzymes classified under EC 3.2.1.1 (α-D-(1→4)-glucan glucanohydrolase), which cleave the α-D-(1→4) O-glycosidic linkages in starch.

The terms “thermostable” and “thermostability,” with reference to an enzyme, refer to the ability of the enzyme to retain activity after exposure to an elevated temperature. The thermostability of an enzyme, such as an amylase enzyme, is measured by its half-life (t½) given in minutes, hours, or days, during which half the enzyme activity is lost under defined conditions. The half-life may be calculated by measuring residual a-amylase activity following exposure to (i.e., challenge by) an elevated temperature.

The terms, “wild-type,” “parental,” or “reference,” with respect to a polypeptide or polynucleotide, refer to a naturally-occurring polypeptide that does not include a man-made substitution, insertion, or deletion at one or more amino acid or nnucleotide positions.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The term “recombinant,” when used in reference to a subject cell, nucleic acid, protein or vector, indicates that the subject has been modified from its native state.

The terms “recovered,” “isolated,” and “separated,” refer to a compound, protein (polypeptides), cell, nucleic acid, amino acid, or other specified material or component that is removed from at least one other material or component with which it is naturally associated as found in nature.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes and synthetic molecules capable of encoding a polypeptide. Nucleic acids may be single stranded or double stranded, and may contain chemical modifications. The terms “nucleic acid” and “polynucleotide” are used interchangeably. Unless otherwise indicated, nucleic acid sequences are presented in 5′-to-3′ orientation.

The terms “transformed,” “stably transformed,” and “transgenic,” used with reference to a cell means that the cell contains a non-native (e.g., heterologous) nucleic acid sequence integrated into its genome or carried as an episome that is maintained through multiple

The term “fermenting organism” refers to any organism, including bacterial and fungal organisms, including yeast and filamentous fungi, suitable for producing a desired fermentation product.

A “host strain” or “host cell” is an organism into which an expression vector, phage, virus, or other DNA construct, including a polynucleotide encoding a polypeptide of interest (e.g., an amylase) has been introduced. The term “host cell” includes protoplasts created from cells.

The term “filamentous fungi” refers to all filamentous forms of the subdivision Eumycotina, particulary Pezizomycotina species.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

“Fused” polypeptide sequences are connected, i.e., operably linked, via a peptide bond between two subject polypeptide sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

The term “specific activity” refers to the number of moles of substrate that can be converted to product by an enzyme or enzyme preparation per unit time under specific conditions. Specific activity is generally expressed as units (U)/mg of protein.

“Percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

The phrase “simultaneous saccharification and fermentation (SSF)” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

The term “fermented beverage” refers to any beverage produced by a method comprising a fermentation process, such as a microbial fermentation, e.g., a bacterial and/or fungal fermentation. “Beer” is an example of such a fermented beverage, and the term “beer” is meant to comprise any fermented wort produced by fermentation/brewing of a starch-containing plant material.

The term “malt” refers to any malted cereal grain, such as malted barley or wheat.

The term “wort” refers to the unfermented liquor run-off following extracting the grist during mashing.

The term “about” refers to ±15% to the referenced value.

### 2. Variant Lipase Polypeptides

An aspect of the present compositions and methods are variant lipase molecules that include combinations of mutations that improve their performance in controlling antifoaming in a fermentation process.

The variant lipases, and methods of use, thereof, are derived from Thermomyces lanuginosus lipase (TLL; see, e.g., NCBI Accession Nos. O59952.1, AOE45082.1, 1DT3_A and 1GT6_A), represented by SEQ ID NO: 1, below:

EVSQDLFNQFNLFAQYSAAAYCGKNNDAPAGTNITCTGNACPEVEKADAT
ELYSFEDSGVGDVTGFLALDNTNKLIVLSFRGSRSIENWIGNLNFDLKEI
NDICSGCRGHDGFTSSWRSVADTLRQKVEDAVREHPDYRVVFTGHSLGGA
LATVAGADLRGNGYDIDVFSYGAPRVGNRAFAEFLTVQTGGTLYRITHTN
DIVPRLPPREFGYSHSSPEYWIKSGTLVPVTRNDIVKIEGIDATGGNNQP
NIPDIPAHLWYFGLIGTCL

The variant lipases include one or more of the substitutions G91A, D96W and E99K, with reference to SEQ ID NO: 1 (see, e.g., SEQ ID NO: 2 in WO 2003/099016 A2). In some embodiments, the variant lipases included all three of the substitutions G91A, D96W and E99K.

The variant lipases are fusion proteins and further include a portion of the C-terminus of Fusarium oxysporum lipase (FOX; NCBI Accession No. ABR12479.1), represented by SEQ ID NO: 6, below:

MLLLPLLSAITLAVASPVALDDYVNSLEERAVGVTTTDFGNFKEYIQHGA
AAYCNSEAAAGSKITCSNNGCPTVQGNGATIVTSFGSKTGIGGYVATDSA
RKEIVVSFRGSINIRNWLTNLDFGQEDCSLVSGCGVHSGFQRAWNEISSQ
ATAAVASARKANPSFKVISTGHSLGGAVAVLAAANLRVGGTPVDIYTYGS
PRVGNVQLSAFVSNQAGGEYRVTHADDPVPRLPPLIFGYRHTTPEFWLSG
GGGDTVDYTISDVKVCEGAANLGCNGGTLGLDIAAHLHYFQATDACNAGG
FSWRRYRSAESVDKRATMTDAELEKKLNSYVQMDKEYVKNNQARS

In the present fusion polypepdes, the portion of the C-terminus of FOX that is fused to the TLL portion of the lipase should not exceed 50 contiguous amino acid residues of the most C-terminal portion of FOX and should not less than 12 contiguous amino acid residues, based in the amino acid sequence of SEQ ID NO: 6. In some embodiments, the portion of the C-terminus of FOX should not exceed 15 contiguous amino acid residues of the most C-terminal portion of FOX and should not less than 12 contiguous amino acid residues. In some embodiments, the portion of the C-terminus of FOX is 12 contiguous amino acid residues of the most C-terminal portion of FOX.

In some embodiment, the C-terminal portion of the TLL portion of the variant lipase may have a small number of fewer residues or a small number of additional residues, for example, as the result of using convenient restriction sites for cloning purposes. In some embodiment, the number of fewer of additional residues is 10 or less, 9 or less, 8 or less, 7 or less 6 or less, 5 or less, 4 or less, 3 or less, 2 or less, or even 1 or less. In a particular embodiment, the number of fewer residues is 7±3, 7±2, 7±1, or exactly ±7. In one particular embodiment, the number of fewer residues is exactly −7.

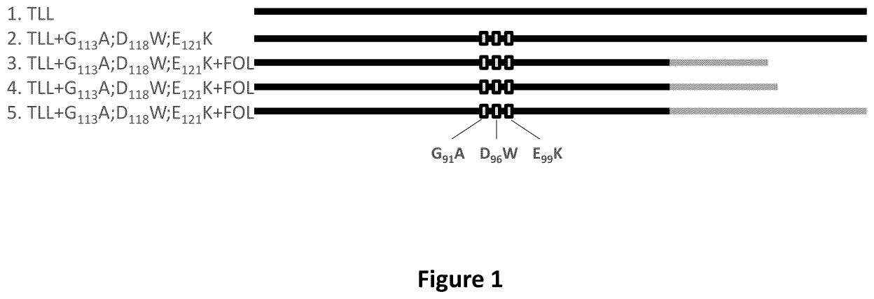

Features of the various lipase molecules are summarized in Table 2 in the Examples and a graphic representation of the molecules is provided in FIG. 1. The amino acid sequence of a particular variant lipase is shown, below, as SEQ ID NO: 4:

EVSQDLFNQFNLFAQYSAAAYCGKNNDAPAGTNITCTGNACPEVEKADAT
ELYSFEDSGVGDVTGFLALDNTNKLIVLSFRGSRSIENWIANLNFWLKKI
NDICSGCRGHDGFTSSWRSVADTLRQKVEDAVREHPDYRVVFTGHSLGGA
LATVAGADLRGNGYDIDVFSYGAPRVGNRAFAEFLTVQTGGTLYRITHTN
DIVPRLPPREFGYSHSSPEYWIKSGTLVPVTRNDIVKIEGIDATGGNNQP
NIPDIPAHLWYFQATDACNAGGFS

LIP5, described, herein, is identical to LECITASE® Ultra, a baking enzyme apparently rebranded as a Defoamer for use in ethanol facilities that also use a thermostable protease. LIP5 serves as a benchmark for the improved lipase variants described, herein. The amino acid sequence of LIP5 is shown, below, as SEQ ID NO: 5:

EVSQDLFNQFNLFAQYSAAAYCGKNNDAPAGTNITCTGNACPEVEKADAT
ELYSFEDSGVGDVTGFLALDNTNKLIVLSFRGSRSIENWIANLNFWLKKI
NDICSGCRGHDGFTSSWRSVADTLRQKVEDAVREHPDYRVVFTGHSLGGA
LATVAGADLRGNGYDIDVFSYGAPRVGNRAFAEFLTVQTGGTLYRITHTN
DIVPRLPPREFGYSHSSPEYWIKSGTLVPVTRNDIVKIEGIDATGGNNQP
NIPDIPAHLWYFQATDACNAGGFSWRRYRSAESVDKRATMTDAELEKKLN
SYVQMDKEYVKNNQARS

In some embodiments, the present lipase variants have the indicated combinations of mutations and a defined degree of amino acid sequence homology/identity to SEQ ID NO: 4, for example, at least 95%, at least 96%, at least 97%, at least 98% or even at least 99% amino acid sequence homology/identity. Preferably, the variant lipase does not have the amino acid sequence of SEQ ID NO: 3 or SEQ ID NO: 5.

The present lipase may include any number of conservative amino acid substitutions. Exemplary conservative amino acid substitutions are listed in Table 1

TABLE 1
Conservative amino acid substitutions

| Original residue | Code | Replace with any of . . . |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, He, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

The reader will appreciate that some of the above mentioned conservative mutations can be produced by genetic manipulation, while others are produced by introducing synthetic amino acids into a polypeptide by genetic or other means.

The present variant lipase may be “precursor,” “immature,” or “full-length,” in which case they include a signal sequence and/or a pro-sequence, or “mature,” in which case they lack a signal sequence. Mature forms of the polypeptides are generally the most useful. Unless otherwise noted, the amino acid residue numbering used herein refers to the mature forms of the respective variant lipase polypeptides. The present lipase variants polypeptides may also be truncated to remove the N or C-termini, so long as the resulting polypeptides retain lipase activity.

### 3. Metal Salts

Any suitable metal salt may be used in combination with the present lipase variants. Preferred metal salts include salts of a metal selected from the group consisting of calcium, magnesium, sodium and potasium. Preferred metal salts include divalent ions, such as CaCl₂, CaCO₃, Ca(OH)₂, Salt including monovalent metal can also be used.

### 4. Uses of the Improved Variant Lipase

The improved antifoaming lipase described herein is preferably used in a fermentation process, which are well known in the art. A fermentation process usually includes liquefaction and saccharification of a raw material comprising starch, e.g., from grain. Any variation of liquefaction or saccharification may be used in combination with the fermentation process of the present invention. For example, liquefaction and saccharification may be carried out simultaneously or in an overlapping manner. Similarly, saccharification and fermentation may be carried out separately or simultaneously, as in the case of simultaneous saccharification and fermentation (SSF).

The raw material for the fermentation processes may in be obtained from tubers, roots, stems, cobs, legumes, cereals or whole grain. More specifically the granular starch may be obtained from corns, cobs, wheat, barley, rye, milo, sago, cassava, tapioca, sorghum, rice, peas, bean, banana or potatoes.

The improved antifoaming lipase variants described, herein, is suitable for application in fermentation processes comprising thermal gelatinization of the milled grain (i.e., a “traditional fermentation” processes) as well as in fermentation processes which does not comprise such a thermal gelatinization (i.e., a “raw starch hydrolysis” or “cold cook” process), in which liquefaction is performed at or below the gelatinization temperature. Traditional fermentation processes wherein the antifoaming system of the present invention may be applied are described in, e.g., WO199628567 and WO200238787. Cold cook processes wherein the antifoaming system of the present invention may be applied are described in, e.g., WO 2003/66816, WO 2003/66826 and WO 2004/080923.

The present lipase, optionally along with other enzymes and metal salts, is preferably added prior to, or early in, fermentation, where foaming is most problematic. Typically, the addition will be sometime during saccharification. In the case of SSF, addition will typically be early during SSF. Addition can even be during liquefaction, so long as the variant lipase is not destroyed by heat. Addition can be simultaneous with yeast addition, and yeast products mixed with the variant lipases, or even yeast expressing the variant lipases, are contemplated.

### EXAMPLES

### Example 1

### Construction of Lipase Expression Vectors

A series of expression vectors were constructed to express codon-optimized Thermomyces lanuginosus lipase (TLL; SEQ ID NO: 1) and variants, thereof, in Trichoderma reesei. FIG. 1 shows the features of the parent molecules and the variants, including the mutations relative to parental TLL. For numbering and nomenclauture convenience LIP1 is wild-type TLL (i.e., SEQ ID NO: 1) and LIP2-LIP5 (SEQ ID NOs: 2-5) are the variants. All four variants included the substitutions G91A, D96W and E99K (see, e.g., SEQ ID NO: 2 in WO 2003/099016 A2). LIP3-LIP5 further include a small truncation of the C-terminus of TLL and fusion to various length of the C-termus of Fusarium oxysporum lipase (FOX; SEQ ID NO: 6).

Genes encoding the variants were made using standard molecular biology techniques based on the codon optimized sequence of SEQ ID NO: 7. All genes were under control of the transcriptional control of (i.e., operably linked to) the native T. reesei cbh1 promoter and terminator. The expression vectors included the pyr2 selectable marker (encoding orotate phosphoribosyl transferase) upstream of the cbh1 promoter and the TLL gene.

Features of the various lipase molecules are summarized in Table 2 and a graphic representation of the molecules is shown in FIG. 1. Note that LIP5 is identical to LECITASE® Ultra, a baking enzyme apparently rebranded as PROTREAT™ Defoamer for use in ethanol facilities that also use a thermostable protease.

TABLE 2
Features of TLL lipase and variants, thereof

| | Features | | Mature | TLL | FOX | |
|---|---|---|---|---|---|---|
| Molecule | (relative to TL) | Immature length | length | length | length | SEQ ID NO |
| LIP1 | Wild-type TLL | 291 | 269 | 269 | 0 | 1 |
| LIP2 | G91A, D96W, E99K | 291 | 269 | 269 | 0 | 2 |
| LIP3 | G91A, D96W, E99K, | 295 | 273 | 262 | 11 | 3 |
| | C-termimnal 7 residues | | | | | |
| | truncated, 11 residues of | | | | | |
| | C-termus from FOX | | | | | |
| LIP4 | G91A, D96W, E99K, | 296 | 274 | 262 | 12 | 4 |
| | C-termimnal 7 residues | | | | | |
| | truncated, 12 residues of | | | | | |
| | C-termus from FOX | | | | | |
| LIP5 | G91A, D96W, E99K, | 341 | 319 | 262 | 55 | 5 |
| | C-termimnal 7 residues | | | | | |
| | truncated, 55 residues of | | | | | |
| | C-termus from FOX | | | | | |

The amino acid sequences of LIP1-LIP5 and of FOX, and the nucleotide sequence of the codon-optimized gene encoding TLL are shown below. Note that the SEQ ID NOs refer to mature polypeptide sequences. (i.e., without signal seuences) unless otherwise specified.

The mature amino acid sequence of LIP1 is shown, below, as SEQ ID NO: 1:

EVSQDLFNQFNLFAQYSAAAYCGKNNDAPAGTNITCTGNACPEVEKADAT
ELYSFEDSGVGDVTGFLALDNTNKLIVLSFRGSRSIENWIGNLNFDLKEI
NDICSGCRGHDGFTSSWRSVADTLRQKVEDAVREHPDYRVVFTGHSLGGA
LATVAGADLRGNGYDIDVFSYGAPRVGNRAFAEFLTVQTGGTLYRITHTN
DIVPRLPPREFGYSHSSPEYWIKSGTLVPVTRNDIVKIEGIDATGGNNQP
NIPDIPAHLWYFGLIGTCL

The mature amino acid sequence of LIP2 is shown, below, as SEQ ID NO: 2:

EVSQDLFNQFNLFAQYSAAAYCGKNNDAPAGTNITCTGNACPEVEKADAT
ELYSFEDSGVGDVTGFLALDNTNKLIVLSFRGSRSIENWIANLNFWLKKI
NDICSGCRGHDGFTSSWRSVADTLRQKVEDAVREHPDYRVVFTGHSLGGA
LATVAGADLRGNGYDIDVFSYGAPRVGNRAFAEFLTVQTGGTLYRITHTN
DIVPRLPPREFGYSHSSPEYWIKSGTLVPVTRNDIVKIEGIDATGGNNQP
NIPDIPAHLWYFGLIGTCL

The mature amino acid sequence of LIP3 is shown, below, as SEQ ID NO: 3:

EVSQDLFNQFNLFAQYSAAAYCGKNNDAPAGTNITCTGNACPEVEKADAT
ELYSFEDSGVGDVTGFLALDNTNKLIVLSFRGSRSIENWIANLNFWLKKI
NDICSGCRGHDGFTSSWRSVADTLRQKVEDAVREHPDYRVVFTGHSLGGA
LATVAGADLRGNGYDIDVFSYGAPRVGNRAFAEFLTVQTGGTLYRITHTN
DIVPRLPPREFGYSHSSPEYWIKSGTLVPVTRNDIVKIEGIDATGGNNQP
NIPDIPAHLWYFQATDACNAGGF

The mature amino acid sequence of LIP4 is shown, below, as SEQ ID NO: 4:

EVSQDLFNQFNLFAQYSAAAYCGKNNDAPAGTNITCTGNACPEVEKADAT
ELYSFEDSGVGDVTGFLALDNTNKLIVLSFRGSRSIENWIANLNFWLKKI
NDICSGCRGHDGFTSSWRSVADTLRQKVEDAVREHPDYRVVFTGHSLGGA
LATVAGADLRGNGYDIDVFSYGAPRVGNRAFAEFLTVQTGGTLYRITHTN
DIVPRLPPREFGYSHSSPEYWIKSGTLVPVTRNDIVKIEGIDATGGNNQP
NIPDIPAHLWYFQATDACNAGGFS

The mature amino acid sequence of LIP5 is shown, below, as SEQ ID NO: 5:

EVSQDLFNQFNLFAQYSAAAYCGKNNDAPAGTNITCTGNACPEVEKADAT
ELYSFEDSGVGDVTGFLALDNTNKLIVLSFRGSRSIENWIANLNFWLKKI
NDICSGCRGHDGFTSSWRSVADTLRQKVEDAVREHPDYRVVFTGHSLGGA
LATVAGADLRGNGYDIDVFSYGAPRVGNRAFAEFLTVQTGGTLYRITHTN
DIVPRLPPREFGYSHSSPEYWIKSGTLVPVTRNDIVKIEGIDATGGNNQP
NIPDIPAHLWYFQATDACNAGGFSWRRYRSAESVDKRATMTDAELEKKLN
SYVQMDKEYVKNNQARS

Mature amino acid sequence of FOX lipase, NCBI Accession No. ABR12479.1 (SEQ ID NO: 6):

MLLLPLLSAITLAVASPVALDDYVNSLEERAVGVTTTDFGNFKEYIQHGA
AAYCNSEAAAGSKITCSNNGCPTVQGNGATIVTSFGSKTGIGGYVATDSA
RKEIVVSFRGSINIRNWLTNLDFGQEDCSLVSGCGVHSGFQRAWNEISSQ
ATAAVASARKANPSFKVISTGHSLGGAVAVLAAANLRVGGTPVDIYTYGS
PRVGNVQLSAFVSNQAGGEYRVTHADDPVPRLPPLIFGYRHTTPEFWLSG
GGGDTVDYTISDVKVCEGAANLGCNGGTLGLDIAAHLHYFQATDACNAGG
FSWRRYRSAESVDKRATMTDAELEKKLNSYVQMDKEYVKNNQARS

The nuceotide sequence of the codon optimized gene encoding LIP1 (i.e., TLL) is shown, below, as SEQ ID NO: 7 (start codon underlined):

CACAAGTTTGTACAAAAAAGCAGGCTCCGCGCCACCATGCGCAGCTCCCT
TGTTCTGTTCTTCGTCAGCGCGTGGACGGCCTTGGCCTCCCCTATTCGTC
GAGAGGTCTCGCAAGATCTGTTCAACCAGTTCAATCTCTTCGCTCAGTAT
TCTGCAGCCGCCTACTGCGGAAAGAACAACGACGCCCCCGCTGGTACCAA
CATCACGTGCACGGGCAACGCCTGCCCCGAGGTCGAGAAGGCGGACGCCA
CGTTTCTCTACTCGTTCGAGGACAGCGGCGTGGGCGATGTCACCGGCTTC
CTGGCTCTCGACAACACGAACAAGCTCATCGTCCTCTCTTTCCGCGGCAG
CCGGTCCATCGAGAACTGGATCGGCAACCTTAACTTCGACCTCAAGGAGA
TCAACGACATCTGCTCCGGCTGCCGCGGCCACGACGGCTTCACTTCGTCC
TGGAGGAGCGTCGCCGACACGCTGCGCCAGAAGGTGGAGGACGCTGTGCG
CGAGCATCCCGACTACCGCGTTGTTTTTACCGGACACAGCCTCGGTGGTG
CGCTCGCTACTGTTGCCGGAGCCGACCTGCGCGGCAATGGGTACGACATC
GACGTGTTCAGCTATGGCGCCCCCCGAGTCGGAAACCGCGCTTTCGCCGA
GTTCCTGACCGTCCAGACCGGCGGCACTCTCTACCGCATCACCCACACCA
ACGATATTGTCCCTCGCCTCCCCCCGCGCGAATTCGGTTACAGCCACTCT
AGCCCCGAGTACTGGATCAAGTCTGGCACCCTCGTCCCCGTCACCCGAAA
CGACATCGTGAAGATCGAGGGCATCGATGCCACCGGCGGCAACAACCAGC
CTAACATTCCGGACATCCCTGCGCACCTGTGGTACTTCGGTCTGATCGGT
ACCTGTCTTTGAGCGCGCCGACCCAGCTTTCTTGTACAAAGT

### Example 2

### Protoplast Preparation and Transformation

Spores of Trichoderma were inoculated into 50 mL of YEG culture medium (5 g/L yeast extract, 20 g/L glucose) and grown in a 250-mL shake flask overnight at 28° C., 180 rpm in a shaker incubator with a 50 mm throw. Germinated spores were collected by a 10-min centrifugation (3,000 g) and washed twice with 10 mL of 1.2 MgSO₄, 10 mM Na-phosphate (pH 5.8). Pellets were resuspended in 40 mL of the same buffer supplemented with 1.2 g of lysing enzymes (Sigma, St Louis, MO) and incubated at 28° C. in a shaker incubator at 100-200 rpm until protoplasts formed. Suspensions were filtered through MIRACLOTH™ (Millipore-Sigma) to remove mycelia and an equal volume of 0.6 M sorbitol, 0.1 M Tris-HCl (pH 7.0) was gently added on top of the protoplast solutions, which were centrifuged at 4,000 rpm for 15 min. Protoplasts were collected from the interphase regions and transferred to new tubes. An equal volume of 1.2 M sorbitol, 10 mM CaCl₂, 10 mM Tris-HCl (pH 7.5) was added and protoplasts were pelleted at 4,000 rpm in 15 min (4° C.) and washed with 1.2 M sorbitol, 10 mM CaCl₂, 10 mM Tris-HCl (pH 7.5). Finally, protoplasts were resuspended in the same buffer to a concentration of 1×10⁸ protoplasts/mL and per 200 μL of protoplasts 50 μL of 25% PEG 6000, 50 mM CaCl₂, 10 mM Tris-HCl (pH 7.5) was added, and the resulting suspensions were stored at −80° C.

PCR products containing the genes described in Example 1 were used to transform the protoplasts. If REMI was used, 5-20 units of a restriction endonuclease were added along with the DNA. 5-20 μg of DNA was added to 200 μL of protoplasts and incubated on ice for 20 min. Afterwards, transformation mixtures were transferred to room temperature and 2 mL of 25% PEG 6,000, CaCl₂, 10 mM Tris-HCl (pH 7.5) and 4 mL of 1.2 M sorbitol, 10 mM CaCl₂, 10 mM Tris-HCl (pH 7.5) was added.

Transformants were selected for uridine prototrophy on AmdS medium supplemented with 10 mM NH₃Cl. For making this medium, a 2×AmdS solution (30 g/L KH₂PO₄, 20 mM acetamide, 1.2 g/L MgSO₄·7H₂O, 1.2 g/L CaCl₂·2H₂O, 0.48 g/L citric acid·H₂O, 0.5 g/L FeSO₄·7H₂O, 40 mg/L ZnSO₄·7H₂O, 8 mg/L CuSO₄·5H₂O, 3.5 mg/L MnSO₄·H₂O, 2 mg/L H₃BO₃ (boric Acid), 40 g/L glucose (pH 4.5) was mixed with an equal volume of 4% agar containing 2 M sorbitol. Other minimal media lacking uridine would also be suitable.

### Example 3

### Expression and Characterization of TLL Molecules

Expression of proteins in suspended Trichoderma cultures has been described. Transformants expressing the various TLL molecules from Example 1 were inoculated in conventional Trichoderma fermentation medium and standard fermentations were performed.

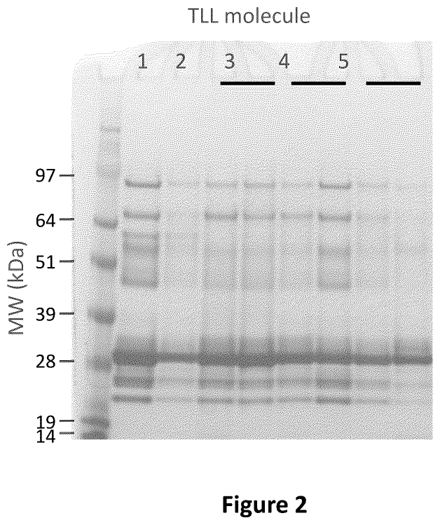

Ferrmentation samples were analyzed for expression levels of lipase by means of SDS-PAGE analysis and using lipase activity assays. An image of a Coomassie-stained SDS-PAGE gel is shown in FIG. 2. The horizontal lines under LIP3-LIP5 indicate that two transformants were grown requiring two lanes of the gel. The expression levels of LIP1, LIP3 and LIP4 were slightly higher than for LIP5.

Total protein (TP) production, phospholipase activity and specific activity were measured in submerged fermentation cultures growing at 28 C, pH 5.75-6.0, with a sugar feed rate of 0.06 g glucose/g DCW/hr.

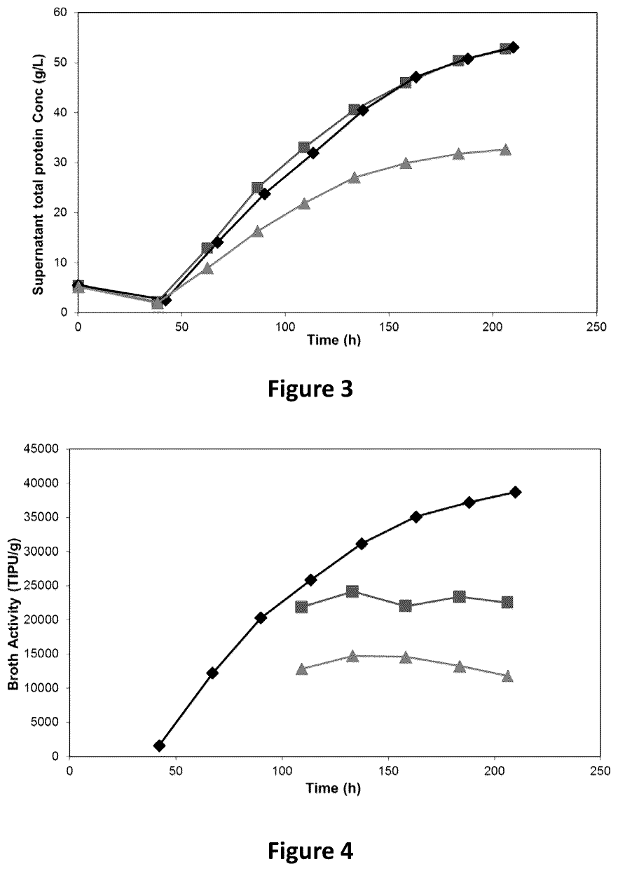

The total protein concentration in the supernatants of culture broth from cells expressing LIP3-LIP5 was measured using the Biuret method with BSA as standard and is shown in the graph in FIG. 3. Phospholipase activity was assayed using L-α-phosphatidylcholine (Avanti 441601G, Avanti Polare Lipids, USA) as a substrate dissolved in 50 mM HEPES buffer with 5 mM CaCl₂ using Triton-X 100 as emulsifier. The amount of free fatty acid liberated during the enzymatic reaction was measured using the NEFA kit (WakoChemicals GmbH, Germany). The results are reported as titratable phospholipase unit (TIPU), which refers to the amount of enzyme that liberates 1 μmol free fatty acid equivalent (FFAeq) per minute at 30° C. and pH 7.0. The result are shown in the graph in FIG. 4.

The amount of lipase in the sample as a fraction of TP was quantified based on the density ratio of bands on a Commassie Blue-stained SDS-PAGE gel analyzed using gel analysis module in ImageJ software. The specific activity versus lipase protein is calculated and summarized Table 3.

TABLE 3
Example of analysis of lipase expression and specific activity

| | Lipase molecule | | |
|---|---|---|---|
| Measurement | LIP3 | LIP4 | LIP5 |
| Lipase vs. TP (%) based on | 76 | 88 | 78 |
| SDS-PAGE gel analysis | | | |
| Activity in broth (TIPU/g) | 24237 | 46610 | 14746 |
| Total protein in broth (g/kg) | 34 | 45 | 18 |
| Lipase in broth (g/kg) | 26 | 40 | 14 |
| Activity/lipase (TIPU/mg | 932 | 1169 | 1068 |
| protein) | | | |

The expression of LIP4 was better than LIP3 and LIP5, as was the activity in broth and the specific activity (see, e.g., FIG. 4 and Table 3).

### Example 4

### Antifoaming Performance of TLL Variants

The effect of lipase molecules LIP2-LIP5 on foam formation and ethanol production was tested in lab scale simultaneous saccharification and fermentation (SSF) using protease-treated and non-treated liquefact. Since LIP1 has previously been shown to be a poor defoaming enzyme (data not shown), is was not included in the experiments. LIP5, which represents a commercial product, was used as a benchmark.

Corn kernels (Arie Blok Animal Nutrition, NL-3440 AA Woerden, Artnr. 377) were milled using a Retsch ZM200 grinding machine with a 3 mm screen at 1,0000 rpm. The resulting corn flour was used to generate 2 kg slurry batches at 34% dry solids by adding tap water to the flour. The pH of the slurry was adjusted to pH 5.1 with H₂SO₄ An α-amylase-containing product (SPEZYME® RSL, DuPont) was added at a commercially relevant dose, followed by addition of thermostable protease ME-3 (WO 2018/118815) to a final concentration of 4 ug/g DS. The mixture was incubated for 2 h at 85° C. with overhead stirring. A control sample was generated where no protease was added to the liquefaction slurry. Following incubation, the treated material (liquefact) was used for subsequent SSF experiments.

The pH of the liquefact was adjusted to pH 4.8 with H₂SO₄. Urea and a glucoamylase product (SYNERXIA® PRIME LC, DuPont Industrial Biosciences) were added at commercially relevant doses. 0.1% w/w dry active yeast (SYNERXIA® PRIME ADY; DuPont Industrial Biosciences) was used for fermentation. The fermentations had zero or 0.1 SAPU/g DS of acid fungal protease (FERMGEN™ 2.5× (DuPont Industrial Biosciences), herein abbreviated AFP).

Defoamer lipases were added to SSF at a dose of 0.5 ug/g DS. The SSF mixture was apportioned into 250 mL polypropylene graded cylinders. The cylinders, provided with foam stoppers, were placed in water baths at 32° C. and stirred magnetically at 350 rpm.

After several hours of fermentation, foam started accumulating at the top of the SSF mixtures leaving traces on the cylinder wall which remained visible even after the foam had collapsed. The level of foam generated during SSF was recorded after 16 h of incubation and expressed volumetrically. The level reduction for duplicate samples are shown in Table 3.

TABLE 3
Summary of relative foam levels measured
after 16 h in different SSF mixtures

| ME-3 | Urea | | AFP | Relative foam |
|---|---|---|---|---|
| (ug/g DS) | (ppm) | TLL molecule | (SAPU/g DS) | (%) |
| 4 | 520 | none | 0.1 | -100- |
| 4 | 350 | LIP2 | -0- | 96 |
| 4 | 350 | LIP3 | -0- | 54 |
| 4 | 350 | LIP4 | -0- | 40 |
| 4 | 350 | LIP5 | -0- | 52 |
| -0- | 520 | none | 0.1 | -100- |
| -0- | 520 | LIP3 | 0.1 | 99 |
| -0- | 520 | LIP4 | 0.1 | 65 |
| -0- | 520 | LIP5 | 0.1 | 81 |

The results show that the addition of lipase has a positive effect on controlling the foam levels of the fermentation system, even in cases where increased urea was used and acid protease was added to the SSF mixture. LIP4 demonstrated the lowest levels of foam in both the presence and absence of thermostable protease during SSF.

### Example 5

### Stability of TLL Variants

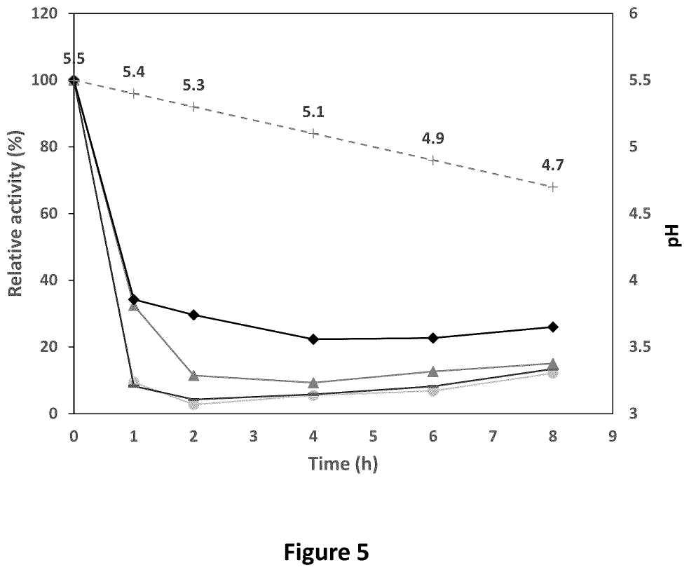

The stability of LIP4, LIP5 and an unrelated commercially-available lipase and truncated variant, thereof, was determined under SSF conditions. Briefly, a 50 mL volume of SSF substrate representing corn liquefact obtained from corn flour and tap water as described in Example 4 at an unadjusted pH 5.5 was incubated with glucoamylase (DISTILASE® XP, DuPont) at a commercially relevant dose and 0.1% w/w dry active yeast (ETHANOL RED® yeast; Lesaffre Advanced Fermentations) in the presence of 352 ppm urea at 33° C. in an orbital shaker at 150 rpm. LIP4, LIP5 and the other lipases were dosed at 0.9 TIPU/g DS. Samples of the fermentation broth were drawn periodically, subjected to centrifugation at 12,000×g to pellet insoluble material, and the supernatant used to test residual lipase activity. As shown in the graph in FIG. 5, LIP4 was clearly more stable than the other molecules tested.

What is claimed is:

1. A variant Thermomyces lanuginosus lipase having at least 95%, optionally at least 98% and optionally at least 99% amino acid sequence identity to the amino acid sequence of SEQ ID NO: 4 and having improved defoaming activity in a fermentation process compared to a reference lipase having the amino acid sequence of SEQ ID NO: 5, wherein the variant lipase comprises: substantially the entire contiguous amino acid sequence of T. lanuginosus lipase, including the the N-terminus, having one or more substitutions selected from the group consisting of G91A, D96W and E99K, with reference to SEQ ID NO; 4, the substantially the entire contiguous amino acid sequence of T. lanuginosus lipase existing as a fusion protein with a contiguous amino acid sequence from Fusarium oxysporum lipase having the amino acid sequence of SEQ ID NO: 2, where the variant lipase has, as its C-terminus, at least 12 but fewer than 55 amino acid residues derived from the C-terminus of F. oxysporum lipase, and wherein the variant lipase does not have the amino acid sequence of SEQ ID NO: 3 or SEQ ID NO: 5.

2. The variant lipase of claim 1 having, as its C-terminus, at least 12 but fewer than 15 amino acid residues derived from the C-terminus of F. oxysporum.

3. The variant lipase of claim 1 or 2 having, as its C-terminus, 12 amino acid residues derived from the C-terminus of F. oxysporum.

4. The variant lipase of any of claims 1-3 having the substitutions G91A, D96W and E99K.

5. The variant lipase of any of claims 1-4 having a small number of fewer or additional residues at the C-terminus of the contiguous amino acid sequence of T. lanuginosus lipase.

6. The variant lipase of any of claims 1-4 having a truncation of residues at the C-terminus of the contiguous amino acid sequence of T. lanuginosus lipase.

7. The variant lipase of any of claims 1-6 having the amino acid sequence of SEQ ID NO: 4.

8. The variant lipase of any of claims 1-7 wherein the fermentation process in which the variant lipase has improved defoaming activity in simultaneous sachharification and fermentation.

9. An improved method for reducing foaming in an ethanol production process using a carbohydrate substrate as feedstock, comprising adding before or during a fermentation step the variant lipase of any of claims 1-7 having improved defoaming activity in a fermentation process compared to the reference lipase having the amino acid sequence of SEQ ID NO: 5.

10. The improved method of claim 9, wherein the fermentation process is saccharification and/or fermentation.

11. The improved method of claim 9 or 10, wherein the fermentation process is simultaneous sachharification and fermentation.

12. A variant Thermomyces lanuginosus lipase having at least 95%, optionally at least 98% and optionally at least 99% amino acid sequence identity to the amino acid sequence of SEQ ID NO: 4 and having improved expression in a Trichoderma host compared to a reference lipase having the amino acid sequence of SEQ ID NO: 5, wherein the variant lipase comprises: substantially the entire contiguous amino acid sequence of T. lanuginosus lipase, including the the N-terminus, having one or more substitutions selected from the group consisting of G91A, D96W and E99K, with reference to SEQ ID NO; 4, the substantially the entire contiguous amino acid sequence of T. lanuginosus lipase existing as a fusion protein with a contiguous amino acid sequence from Fusarium oxysporum lipase having the amino acid sequence of SEQ ID NO: 2, where the variant lipase has, as its C-terminus, at least 12 but fewer than 55 amino acid residues derived from the C-terminus of F. oxysporum lipase, and wherein the variant lipase does not have the amino acid sequence of SEQ ID NO: 3 or SEQ ID NO: 5.

13. The variant lipase of claim 12 having, as its C-terminus, at least 12 but fewer than 15 amino acid residues derived from the C-terminus of F. oxysporum.

14. The variant lipase of claim 12 or 13 having, as its C-terminus, 12 amino acid residues derived from the C-terminus of F. oxysporum.

15. The variant lipase of any of claims 12-14 having the substitutions G91A, D96W and E99K.

16. The variant lipase of any of claims 12-15 having a small number of fewer or additional residues at the C-terminus of the contiguous amino acid sequence of T. lanuginosus lipase.

17. The variant lipase of any of claims 12-16 having a truncation of residues at the C-terminus of the contiguous amino acid sequence of T. lanuginosus lipase.

18. The variant lipase of any of claims 12-17 having the amino acid sequence of SEQ ID NO: 4.

19. The variant lipase of any of claims 11-18 wherein the fermentation process in which the variant lipase has improved defoaming activity in simultaneous sachharification and fermentation.
